(19) United States
(12) Patent Application Publication
(10) Pub. No.: US 2026/0295299 A1
(43) Pub. Date: Oct. 1, 2026

(54) AUTOMATED TARGET PLACEMENT FOR LATTICE RADIATION THERAPY

(71) Applicant: Mayo Foundation for Medical Education and Research, Rochester, MN (US)

(72) Inventors: Christopher L. Deufel, Rochester, MN (US); Douglas J. Moseley, Rochester, MN (US); Michael P. Grams, Rochester, MN (US)

(21) Appl. No.: 19/479,516

(22) PCT Filed: Apr. 29, 2024
(86) PCT No.: PCT/US2024/026861

§ 371 Date: Oct. 28, 2025

Related U.S. Application Data
(60) Provisional application No. 63/499,108, filed on Apr. 28, 2023.

(51) Int. Cl. A61N 5/10

(52) U.S. Cl. CPC A61N 5/1042; A61N 5/1031; A61N 2005/1034

(57) ABSTRACT

Placement of dose vertices in a dose lattice for lattice radiation treatment (LRT) is automated using a series of dose vertex constraints and objectives. The dose vertex constraints place limitations on the spacing and other spatial arrangement of dose vertices in the dose lattice, relative to other dose vertices, tumor volume, and/or organs-at-risk. The dose vertex objectives give preference to spatial arrangements of dose vertices in the dose lattice, such as having more centrally located dose vertices.

### CROSS-REFERENCE TO RELATED APPLICATIONS

This application claims the benefit of U.S. Provisional Patent Application Ser. No. 63/499,108, filed on Apr. 28, 2023, and entitled “AUTOMATED TARGET PLACEMENT FOR LATTICE RADIATION THERAPY,” which is herein incorporated by reference in its entirety.

### BACKGROUND

Lattice radiation therapy (LRT) is a type of spatially fractionated radiation therapy. In LRT, the radiation dose is targeted to one or more target contours and/or orientations, which may be referred to as lattice points or dose vertices. The dose vertices are distributed throughout the gross tumor volume (GTV). The collection of dose vertices may be referred to as a dose lattice.

### SUMMARY OF THE DISCLOSURE

It is an aspect of the present disclosure to provide a method for generating a radiation treatment plan for a spatially fractionated radiation treatment, such as lattice radiation treatment. Medical imaging data of a subject are accessed with a computer system. Dose vertex constraint data and dose vertex objective data are also accessed with the computer system. The dose vertex constraint data include spatial constraints on potential dose vertex locations in a dose lattice, and the dose vertex objective data include spatial preferences on potential dose vertex locations in a dose lattice. A tumor region depicted in the medical imaging data is selected using the computer system. A dose lattice is constructed with the computer system by generating and arranging dose vertices in the tumor region subject to the dose vertex constraint data and the dose vertex objective data. The dose lattice is stored, by the computer system, such as in a radiation treatment plan for the subject.

### BRIEF DESCRIPTION OF THE DRAWINGS

FIG. 1 illustrates an example dose lattice composed of a plurality of dose vertices arranged within a tumor region.

FIG. 2 is a flowchart setting forth the steps of an example method for generating a radiation treatment plan for lattice radiation treatment.

FIGS. 3A and 3B illustrate example components of a graphical user interface (“GUI”) that may be used to generate dose vertices to be used in a radiation treatment plan for lattice radiation treatment according to some examples described in the present disclosure.

FIG. 4 is an example workflow for planning a lattice radiation treatment for a subject.

FIG. 5 is a flowchart setting forth the steps of an example method for generating a dose lattice subject to dose vertex constraints and objectives using a Monte Carlo-based technique.

FIG. 6 is a block diagram of an example lattice radiation treatment planning system.

FIG. 7 is a block diagram of example components that can implement the lattice radiation treatment planning system of FIG. 6.

### DETAILED DESCRIPTION

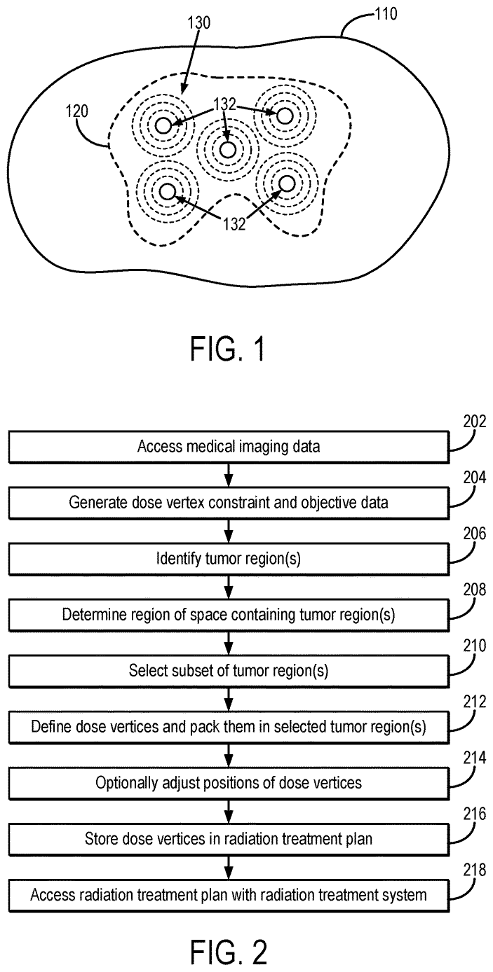

Described here are systems and methods for automating target placement for lattice radiation therapy (LRT). In LRT, a dose lattice is constructed as a three-dimensional (3D) dose distribution containing multiple high-dose regions surrounded by low-dose regions. The high-dose regions are generally shaped as discrete, often spherical, regions of higher dose. These high-dose regions may be referred to as dose vertices. For example, as shown in FIG. 1, a subject 110 has a tumor region 120 identified for treatment. The tumor region may be, for example, a gross tumor volume (GTV). A dose lattice 130 is constructed as a plurality of dose vertices 132 that are arranged within the tumor region 120. In the systems and methods described in the present disclosure, the placement of the radiation targets (i.e., the dose vertices 132) is automated based on a set of objectives and/or constraints.

In volumetric modulated arc lattice radiation therapy (VMAT LRT) treatment planning, the dose is targeted to multiple spherical contours, or dose vertices, which are distributed throughout the GTV and subject to rules governing their separation and distance to adjacent organs at risk (OARs). The disclosed systems and methods automate dose vertex placement in order to improve planning consistency and efficiency. As a non-limiting example, a Monte Carlo-based algorithm (e.g., a Monte Carlo simulation) is used to optimize the number of dose vertices that can be packed into the GTV, subject to positioning constraints and objectives. As another example, the treatment planning algorithm allows for a user to select a preference for solutions where dose vertices are more centrally placed within the tumor region, less peripherally placed within the tumor region, or both, to minimize or otherwise reduce risk of exposure to non-targeted tissues and/or OARs. As another example, dose vertices may be anchored into specific regions of the GTV and additional vertices may be optimized in addition to the anchor points. In some other implementations, the treatment planning algorithm can allow a user to select a preference for solutions where dose vertices are distanced away from selected non-targeted tissues and/or OARs. As still another example, the treatment planning algorithm can allow for a user to select a preference for solutions that minimize how the incident radiation passes through, exits, or scatters dose into selected non-targeted tissues and/or OARs. As yet another example, the treatment planning algorithm can allow for the user to identify dose metrics for the target or non-targeted tissues and/or OARs that establish a preference for dose vertices that optimize the dose metrics. The treatment planning algorithm may, in some instances, also allow the user to adjust the spacing of the vertices in both the axial planes and the longitudinal direction to either increase or decrease the magnitude of the low dose between the vertices. Additionally or alternatively, artificial intelligence (AI) methods can be used to instruct a model (e.g., an AI model) that achieves similar performance to an algorithm or past practice.

Referring now to FIG. 2, a flowchart is illustrated setting forth the steps of an example method for generating a lattice radiation treatment plan by constructing a dose lattice through the automated placement of dose vertices within one or more identified tumor regions.

The method includes accessing medical imaging data of a subject for whom a radiation treatment plan will be developed, as indicated at step 202. The medical imaging data are accessed with a computer system, which may be a part of a radiation treatment planning system. The medical imaging data may be accessed by retrieving previously acquired medical imaging data from a memory or other machine-readable storage device or medium. Additionally or alternatively, the medical imaging data may be accessed by acquiring the medical imaging data with a medical imaging system and transferring the medical imaging data to the computer system. In general, the medical imaging data include medical images of the subject, which are acquired to facilitate identifying tumor regions and OARs.

Dose vertex constraint and objective data are generated by the computer system, as indicated at step 204. The dose vertex constraint and objective data may include constraints and/or objectives for the dose vertices. Dose vertex constraints may include spacing constraints, such as rules indicating that dose vertices should be spaced in a certain way, indicating that dose vertices should be some distance apart from each other, indicating that dose vertices should be some distance away from other areas, and so on. Dose vertex objectives may include desired goals for the dose lattice, such as areas where dose vertices should be preferentially positioned and/or regions where fewer dose vertices should be positioned.

As an example, the dose vertex constraint and objective data may include one or more of dose vertex size constraints, dose vertex shape constraints, constraints on the proximity of a dose vertex to the edge of the tumor region(s), spacing requirements between dose vertices (e.g., an enforced minimum, or otherwise reduced, center-to-center spacing), constraints on the distances of dose vertices from the nearest OAR, constraints on the longitudinal (e.g., z-axis) separation between dose vertices, objectives for prioritizing centrally placing dose vertices in the tumor region(s), objective for deprioritizing placing dose vertices in the periphery of the tumor region(s), objectives for distance of dose vertices from OARs, objectives for the entrance and exit of radiation beamlets through OARs, and so on. As described above, in some instances the dose vertex constraint and/or dose vertex objective data may include user preferences for distances from OAR(s); minimizing radiation dose received by OAR(s); optimizing the relationship between the dose to the targets, the tumor, and the OAR(s); and so on.

Table 1 below illustrates an example set of dose vertex placement constraints and objectives. In this example, the dose vertex placement is subject to four constraints and one objective, where Rᵥₑᵣₜₑₓ: is the dose vertex size (e.g., the radius of the dose vertex).

TABLE 1
Example Dose Vertex Placement Constraints and Objectives

| | Parameter Value |
|---|---|
| Constraint | |
| 1. Minimum distance of dose vertices from the | d_{GTV} ≥ 0.5 cm + Rᵥₑᵣₜₑₓ |
| surface of the GTV (d_{GTV}) | |
| 2. Minimum distance of dose vertices from | d_{OAR} ≥ 1 cm + Rᵥₑᵣₜₑₓ |
| OARs (d_{OAR}) | |
| 3. Minimum center-to-center spacing of dose | d_{center} ≥ 2 cm |
| vertices (d_{center}) | |
| 4. Minimum longitudinal (z-axis) separation | zₛₑₚ ≥ 2 cm, |
| (zₛₑₚ) or Euclidean distance between dose | d_{co-axial} ≥ 6 cm |
| vertices sharing the same z-coordinates | |
| (d_{co-axial}) | |
| Objective | |
| 1. Placement of dose vertices near the center of | μ = 0.2 cm |
| mass of GTV axial slices | |

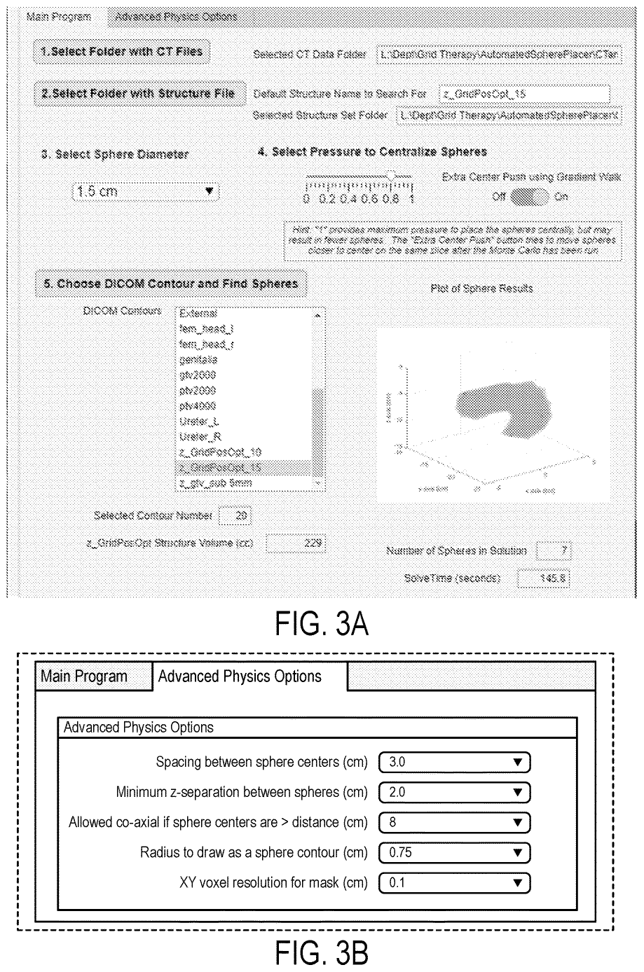

In some implementations, the dose vertex constraint and objective data may be adjusted by a user. For example, the computer system may generate a user interface (e.g., a graphical user interface) that allows for the user to control the size, shape, and spatial relationship (e.g., spacing constraints) between dose vertices. An example graphical user interface is illustrated in FIGS. 3A and 3B.

Additionally or alternatively, the dose vertex constraint and objective data may include dose metrics in the constraints or objectives. For instance, dose metrics may be incorporated by calculating or estimating the dose to the tumor and/or nearby organs as a result of a dose vertex location.

One or more tumor regions are identified with the computer system, as indicated at step 206. As one example, the tumor regions may be identified based on the medical imaging data. The tumor regions may be identified using an AI based approach. As an example, a machine learning algorithm may be used to identify the tumor regions. For instance, a machine learning-based clustering algorithm, such as DBSCAN or HDBSCAN, may be used to identify the tumor regions into which the dose vertices will be placed. Additionally or alternatively, selecting the one or more tumor regions may include fitting a geometric arrangement within a target region and selecting those tumor regions in which the geometric arrangement can be fit.

A region of space containing the identified tumor regions is then determined by the computer system, as indicated at step 208. For example, a region of space that contains the tumor regions may be determined, in part, based on the medical imaging data and the identified tumor regions.

A subset of the tumor regions that comply with the constraints and objectives in the dose vertex constraint and objective data are then determined, as indicated at step 210. For instance, a subset of the tumor regions in which dose vertices will comply with positioning constraints (e.g., dose vertex proximity to tumor edge) may be determined. The construction of the subset of the tumor regions that comply with the constraints and objectives in the dose vertex constraint data and dose vertex objective data may incorporate information regarding the motion of the tumor or OARs during breathing or other patient movement.

Dose vertices are then defined and packed into the selected subset of tumor regions based on the dose vertex constraint and objective data, as indicated at step 212. In some examples, all of the dose vertices will have the same size and shape. In other examples, some or all of the dose vertices may have different sizes, shapes, or both. Thus, the size, shape, and location of each dose vertex may be defined when constructing the dose lattice and/or corresponding dose distribution.

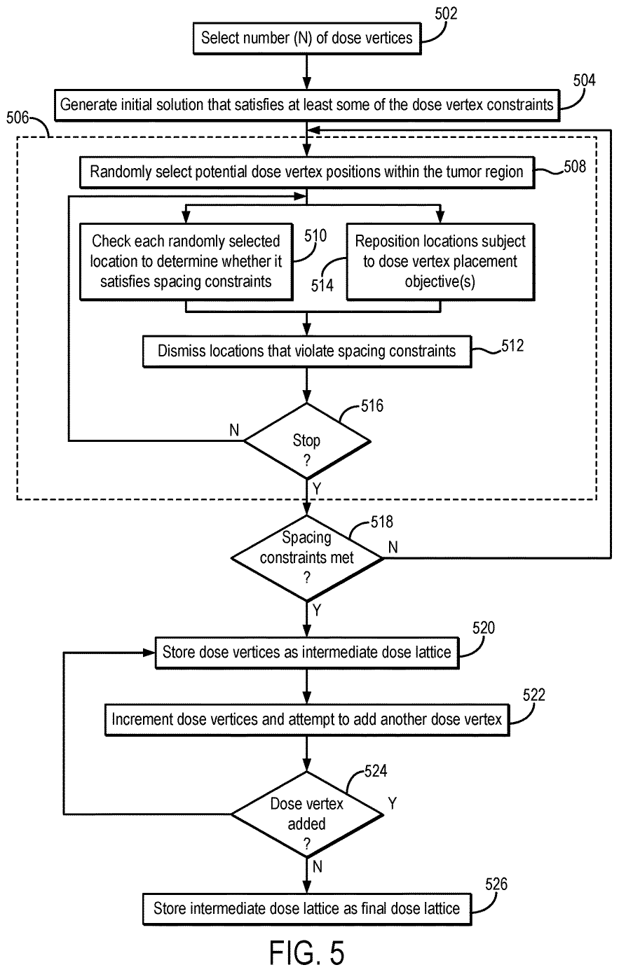

As a non-limiting example, the dose vertices may be defined using an iterative algorithm, such as the one described below in more detail with respect to FIG. 5. Additionally or alternatively, the dose vertices may be positioned within the tumor region(s) using other techniques, including those based on heuristics (e.g., by defining a sequence of instructions based on the dose vertex object data, which will produce desirable dose vertex locations); other optimization techniques (e.g., those based on Monte Carlo, Monte Carlo-like, linear programming, gradient-search, or evolutionary methods and incorporating the dose vertex constraint and objective data); and/or machine learning, deep learning, or other AI methods. For example, a machine learning model may be trained to meet the constraints and objectives in the dose vertex constraint and objective data, such that a tumor region may be input to the machine learning model, and a dose lattice that optimally fills the tumor region subject to the constraints and objectives in the dose vertex constraint and objective data is generated as an output. The machine learning model may be trained, for example on training data including previous clinical plans, whether generated by a human user or an automated process.

Additionally or alternatively, selective pressure may be used to drive solutions for placement of the dose vertices with a desired dose distribution. In some implementations, nomograms may be established and/or used to recommend or assess the proper number of dose vertex locations for a given tumor shape and size.

The defined dose vertices may optionally be further adjusted based on one or more objectives in the dose vertex constraint and objective data, as indicated at step 214. For example, an objective in the dose vertex constraint and objective data may include an objective to centrally position the dose vertices in a tumor region. In these instances, the dose vertices may be adjusted to move them more centrally within the tumor region(s), if possible. For example, the center-of-mass of each slices in which there are dose vertices may be identified. The nearest paths between the each dose vertex location and its corresponding center of mass may be determined. The paths may be divided into multiple steps (e.g., 10 steps) for each dose vertex. An attempt may be made to walk each dose vertex along the path towards the center of mass, one step at a time, proceeding as long as the step does not violate any constraint in the dose vertex constraint and objective data.

The dose vertices are then stored as part of a radiation treatment plan for the subject, as indicated at step 216. For example, the dose vertices may be stored as one or more dose lattices and/or dose distributions in the radiation treatment plan. Additional data may also be stored as part of the radiation treatment plan, including the identified tumor regions (e.g., tumor volumes), OAR volumes, medical imaging data, and the like.

In some examples, multiple dose lattices (e.g., multiple solutions to the dose vertex placement problem) and/or dose distributions may be generated and displayed to a user to compare. In these instances, the user may select a preferred dose lattice for use in the radiation treatment plan. Additionally or alternatively, metrics that report the spatial, dose, or other performance of the dose lattice(s) may be determined and presented to the user (e.g., via a graphical user interface). In still other examples, the dose lattice(s) and/or radiation treatment plan may be compared with past solutions for the same subject to provide quality assurance on the radiation treatment plan.

The radiation treatment plan may then be accessed by a radiation treatment system and used to guide the radiation treatment system to perform lattice radiation treatment on the subject, as indicated at step 218. For instance, the computer system can execute the radiation treatment plan to control operation of the radiation treatment system to deliver therapeutic radiation to the subject according to the determined radiation treatment plan. The computer system may be a part of a radiation treatment planning system and/or the radiation treatment system.

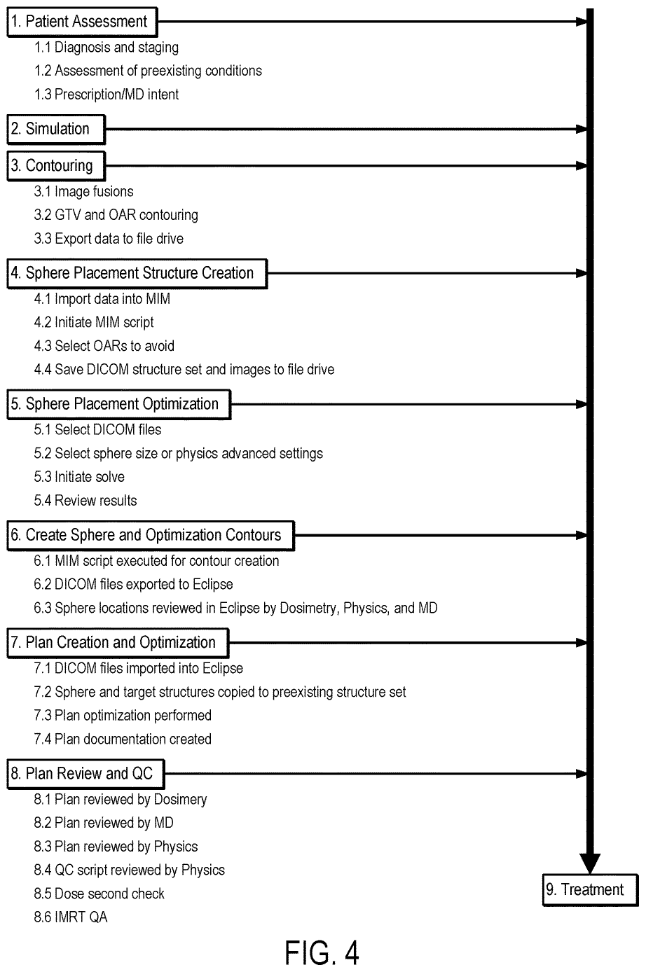

An example radiation treatment planning workflow implementing the methods described in the present disclosure is illustrated in FIG. 4. This process generally includes assessing the patient, a simulation stage, a contouring stage, creating sphere placement structures, optimizing sphere placement, creating sphere and optimization contours, creating and optimizing the radiation treatment plan, reviewing the plan, and delivering treatment to the patient.

In the patient assessment, the patient is diagnosed and staged and any preexisting conditions are assessed by the clinical team. The treatment prescription is then provided. Treatment simulations are then generated based on the prescription. Treatment contouring is then performed. For instance, image fusion can be generated from medical image data acquired from the patient. GTV and OAR contours can then be generated using the image fusions. These data can then be exported for later use and processing.

For sphere placement structure creation, the data may then be imported into a radiation oncology system, such as a MIM system (MIM Software Inc.; Cleveland, OH). Scripts can be executed on the radiation oncology system to select OARs to avoid in the treatment plan. The resulting DICOM structure can be stored for later use. For sphere placement optimization, the DICOM files can be selected and then sphere sizes and/or advanced physics settings can be set. A solution for the radiation treatment plan is then initiated and the results reviewed. Sphere and optimization contours can then be created using the radiation oncology system to execute scripts that create the contour DICOM files. These DICOM files can then be exported to the treatment planning system, such as an Eclipse treatment planning system (Varian Medical Systems, Inc.; Palo Alto, CA). The sphere locations can then be reviewed by the clinical team (e.g., dosimetrist, physicist, physician, etc.) in the treatment planning system. With the DICOM files imported into the treatment planning system, sphere and target structures can be copied to a preexisting structure set. Plan optimization is then performed and the plan documentation created. The plan is then reviewed by the clinical team, the quality control script is reviewed by the physicist, a second dose check is performed, and quality assurance performed. Treatment according to the generated radiation treatment plan can then be initiated.

Referring now to FIG. 5, a flowchart of an example method for iteratively positioning dose vertices within a selected tumor region is shown. The method seeks to pack as many dose vertices as possible into the tumor region, subject to the constraints and/or objectives in the dose vertex constraint and objective data. A number of dose vertices (n=1, 2, . . . , N) to attempt to place within the tumor region is selected, as indicated at step 502. An initial solution that satisfies some, but not all, of the spacing constraints for the dose vertices is generated, as indicated at step 504.

For example, an initial guess of dose vertex positions, P∈P_{possible}, may be selected and used to find a solution for the N dose vertices, where P_{possible} is an array of possible dose vertex locations. The array of possible dose vertex locations, P_{possible}, may be generated using a 3-step workflow. First, the GTV is contracted by an amount (e.g., 0.5 cm) to create GTV_sub_5 mm for the purposes of focusing dose into the interior of the GTV. Second, GTV_sub_5 mm is contracted by Rᵥₑᵣₜₑₓ so that all possible dose vertices may result in spheres that lie entirely inside of GTV_sub_5 mm. Third, any voxels that are closer than Rₛₚₕₑᵣₑ+1 to one or more OARs are deleted. The workflow creates a structure whose contours represent P_{possible}. As a non-limiting example, a DICOM RT Structure and CT files may be accessed by the computer system and a mask may be used to generate the array P_{possible} at the resolution specified by the DICOM CT image set.

For generating the initial guess of the dose lattice and/or dose distribution, the first element in P may be the first element in P_{possible}; that is, P(1)=P_{possible}(1). When solving for more than one dose vertex, N>1, and the initial solution, P, may be constructed by sequentially taking elements from P_{possible} that are at least a distance of d_{center} from the elements already added to P:

$$\{ P ( 2 ) ∈ {P}_{possible} \vert {Δ}_{1} \geq {d}_{center} \vert \} ; \qquad ( 1 )$$

$$⋮$$

$$\{ P ( N ) ∈ {P}_{possible} \vert {Δ}_{1} \geq {d}_{center} … {Δ}_{N - 1} \geq {d}_{center} \vert \}$$

where Δ_{N-1}=P_{possible}−P(N−1). In this example, the initial guess of P satisfies a minimum center-to-center spacing of dose vertices constraint, but may not satisfy other spacing constraints (e.g., constraints specifying a minimum longitudinal separation or Euclidean distance between dose vertices sharing the same z-coordinates).

While not all spacing constraints are met, the dose vertices are repositioned within the tumor region, as generally indicated by process block 506. In one aspect of repositioning the dose vertices, potential dose vertex locations within the tumor region are randomly selected, as indicated at step 508. Each randomly selected location is checked, at step 510, to determine whether it satisfies the spacing constraints. If not, then the potential location is dismissed, as indicated ate step 512.

At the same time, the locations of the dose vertices may be driven to meet one or more of the objectives in the dose vertex constraint and objective data, as indicated at step 514. For instance, the locations of the dose vertices may be driven to meet the central placement objective by disallowing random location choices that are more peripheral, except with some probability. As a non-limiting example, an optimization objective may be implemented to steer the algorithm towards solutions where the dose vertex locations are more centrally located on each axial GTV slice. During each iteration, the new candidate solution is accepted if it satisfies one of the spacing constraints in the dose vertex constraint and objective data (e.g., constraints specifying a minimum longitudinal separation or Euclidean distance between dose vertices sharing the same z-coordinates) and an objective that prefers locations that are closer to the center of the slice, such as:

$$x > 1 - {e}^{- \frac{{Δ}_{COM , d}}{μ}} ; \qquad ( 2 )$$

where x is a random number between 0 and 1; μ is a user-defined preference for central placement with units of centimeters; and Δ_{COM,d} represents the difference in the minimum distance to a center of mass (COM) between the candidate dose vertex location and current location. The parameters Δ_{COM,d} is negative when the candidate location is closer to a COM than the current location. When the candidate location is closer, the expression

$$exp ( - \frac{{Δ}_{COM , d}}{μ} )$$

is greater than 1, and the candidate location is accepted. When the candidate location is further away, the expression

$$exp ( - \frac{{Δ}_{COM , d}}{μ} )$$

is less than 1, and the probability of accepting the candidate location diminishes with increasing Δ_{COM,d}.

The slice COMs may be found by first clustering the elements of P using HDBSCAN, or another suitable hierarchical clustering or other clustering algorithm. The array of COMs may be calculated for of each slice of each cluster. A candidate location that satisfies all of the constraints is chosen if it also satisfies Eqn. (2), which accepts the candidate if its minimum distance to a COM is smaller than the minimum distance from the current location of a COM, and also with a probability that diminishes as the candidate solution is further from the COM.

This random selection process may be repeated until a stopping condition is satisfied, as indicated at decision block 516. For example, the process may repeat for a selected number of iterations (e.g., 100,000 guesses). When a solution that satisfies all of the constraints in the dose vertex constraint and objective data is identified, the search is terminated, as indicated at decision block 518, otherwise the locations of the dose vertices are further adjusted by repeating process block 506.

The dose vertex positions are then stored as an intermediate dose lattice and/or dose distribution, as indicated at step 520. The method may then attempt to add an additional dose vertex to the intermediate dose lattice and/or dose distribution, as indicated at step 522. In these instances, the number of dose vertices is incremented (e.g., N=N+1), and the process outlined above with respect to steps 504-518 is repeated. If the additional dose vertex could not be successfully added, subject to the spacing constraints, as determined at decision block 524, then the intermediate dose lattice and/or dose distribution is stored as the final dose lattice and/or dose distribution, as indicated at step 526. Otherwise, the updated dose lattice and/or dose distribution is stored as the new intermediate dose lattice and/or dose distribution and an additional dose vertex may be added (e.g., by incrementing the dose vertices). If the additional dose vertex cannot be added, then the new intermediate dose lattice and/or dose distribution is stored as the final dose lattice and/or dose distribution, otherwise the intermediate dose lattice and/or dose distribution is again updated and the process of adding an addition dose vertex may be repeated. This process can be repeated until no more dose vertices can be successfully added to the tumor region. In some examples, after the most densely packed solution, Nₘₐₓ, is found, the repositioning process above may be repeated (e.g., with 100,000 more search iterations) to improve the chances of finding a solution where the dose vertex locations are more centrally located on each axial GTV slice.

As a non-limiting example, the dose lattice may be stored as comma separated values indicating the final dose vertex locations along with the total number of dose vertex spheres, the sphere structure names, and sphere diameters. A workflow may be used to generate spherical and concentric shell optimization structures to be used for VMAT LRT treatment planning. This workflow may create individual spherical contours with the specified structure names and expansion diameter as specified in the dose lattice. To facilitate VMAT optimization, the program also creates a structure that is the union of all spheres and concentric shells that are expansions from the surface of the spheres.

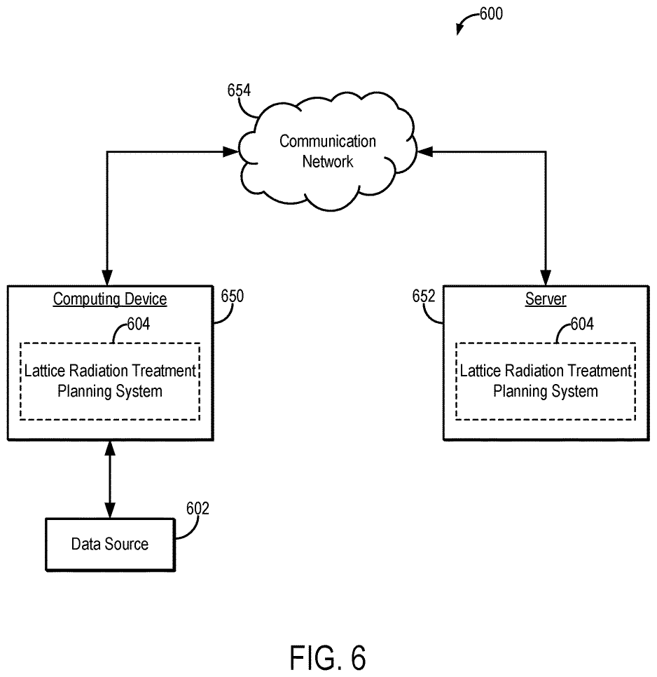

FIG. 6 shows an example of a system 600 for lattice radiation treatment planning in accordance with some embodiments of the systems and methods described in the present disclosure. As shown in FIG. 6, a computing device 650 can receive one or more types of data (e.g., medical imaging data, dose vertex constraint and objective data) from data source 602. In some embodiments, computing device 650 can execute at least a portion of a lattice radiation treatment planning system 604 to generate one or more dose lattices and radiation treatment plans from data received from the data source 602.

Additionally or alternatively, in some embodiments, the computing device 650 can communicate information about data received from the data source 602 to a server 652 over a communication network 654, which can execute at least a portion of the lattice radiation treatment planning system 604. In such embodiments, the server 652 can return information to the computing device 650 (and/or any other suitable computing device) indicative of an output of the lattice radiation treatment planning system 604.

In some embodiments, computing device 650 and/or server 652 can be any suitable computing device or combination of devices, such as a desktop computer, a laptop computer, a smartphone, a tablet computer, a wearable computer, a server computer, a virtual machine being executed by a physical computing device, and so on.

In some embodiments, data source 602 can be any suitable source of data (e.g., measurement data, images reconstructed from measurement data, processed image data, dose vertex constraint and objective data), such as a medical imaging system, another radiation treatment planning system, another computing device (e.g., a server storing measurement data, images reconstructed from measurement data, processed image data, dose vertex constraint and objective data), and so on. In some embodiments, data source 602 can be local to computing device 650. For example, data source 602 can be incorporated with computing device 650 (e.g., computing device 650 can be configured as part of a device for measuring, recording, estimating, acquiring, or otherwise collecting or storing data). As another example, data source 602 can be connected to computing device 650 by a cable, a direct wireless link, and so on. Additionally or alternatively, in some embodiments, data source 602 can be located locally and/or remotely from computing device 650, and can communicate data to computing device 650 (and/or server 652) via a communication network (e.g., communication network 654).

In some embodiments, communication network 654 can be any suitable communication network or combination of communication networks. For example, communication network 654 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 654 can be a local area network, a wide area network, a public network (e.g., the Internet), a private or semi-private network (e.g., a corporate or university intranet), any other suitable type of network, or any suitable combination of networks. Communications links shown in FIG. 6 can each be any suitable communications link or combination of communications links, such as wired links, fiber optic links, Wi-Fi links, Bluetooth links, cellular links, and so on.

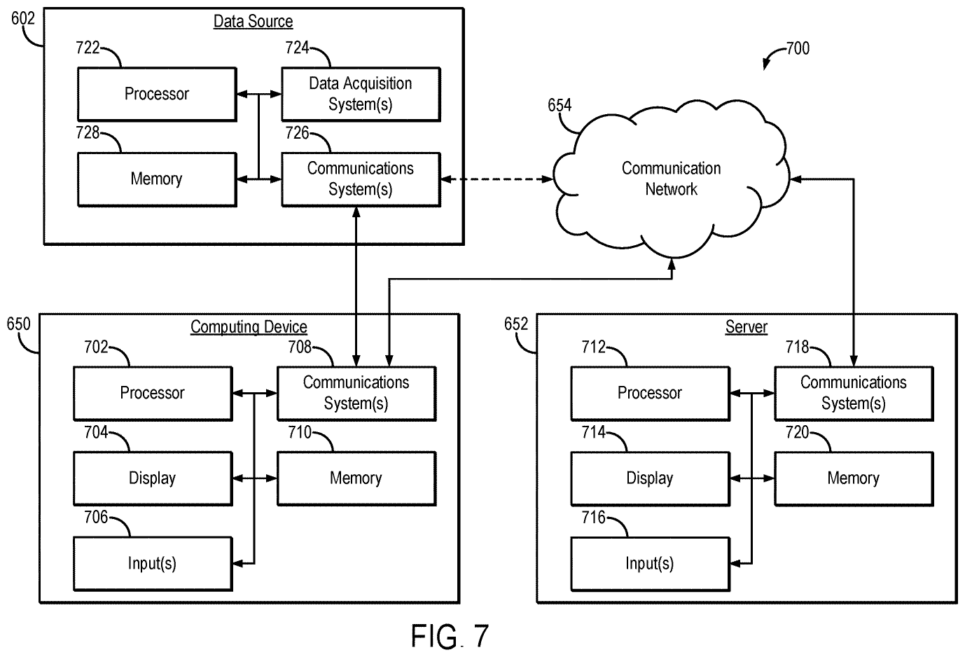

Referring now to FIG. 7, an example of hardware 700 that can be used to implement data source 602, computing device 650, and server 652 in accordance with some embodiments of the systems and methods described in the present disclosure is shown.

As shown in FIG. 7, in some embodiments, computing device 650 can include a processor 702, a display 704, one or more inputs 706, one or more communication systems 708, and/or memory 710. In some embodiments, processor 702 can be any suitable hardware processor or combination of processors, such as a central processing unit (CPU), a graphics processing unit (GPU), and so on. In some embodiments, display 704 can include any suitable display devices, such as a liquid crystal display (LCD) screen, a light-emitting diode (LED) display, an organic LED (OLED) display, an electrophoretic display (e.g., an “e-ink” display), a computer monitor, a touchscreen, a television, and so on. In some embodiments, inputs 706 can include any suitable input devices and/or sensors that can be used to receive user input, such as a keyboard, a mouse, a touchscreen, a microphone, and so on.

In some embodiments, communications systems 708 can include any suitable hardware, firmware, and/or software for communicating information over communication network 654 and/or any other suitable communication networks. For example, communications systems 708 can include one or more transceivers, one or more communication chips and/or chip sets, and so on. In a more particular example, communications systems 708 can include hardware, firmware, and/or software that can be used to establish a Wi-Fi connection, a Bluetooth connection, a cellular connection, an Ethernet connection, and so on.

In some embodiments, memory 710 can include any suitable storage device or devices that can be used to store instructions, values, data, or the like, that can be used, for example, by processor 702 to present content using display 704, to communicate with server 652 via communications system(s) 708, and so on. Memory 710 can include any suitable volatile memory, non-volatile memory, storage, or any suitable combination thereof. For example, memory 710 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some embodiments, memory 710 can have encoded thereon, or otherwise stored therein, a computer program for controlling operation of computing device 650. In such embodiments, processor 702 can execute at least a portion of the computer program to present content (e.g., images, user interfaces, graphics, tables), receive content from server 652, transmit information to server 652, and so on. For example, the processor 702 and the memory 710 can be configured to perform the methods described herein (e.g., the method of FIG. 2, the method of FIG. 5).

In some embodiments, server 652 can include a processor 712, a display 714, one or more inputs 716, one or more communications systems 718, and/or memory 720. In some embodiments, processor 712 can be any suitable hardware processor or combination of processors, such as a CPU, a GPU, and so on. In some embodiments, display 714 can include any suitable display devices, such as an LCD screen, LED display, OLED display, electrophoretic display, a computer monitor, a touchscreen, a television, and so on. In some embodiments, inputs 716 can include any suitable input devices and/or sensors that can be used to receive user input, such as a keyboard, a mouse, a touchscreen, a microphone, and so on.

In some embodiments, communications systems 718 can include any suitable hardware, firmware, and/or software for communicating information over communication network 654 and/or any other suitable communication networks. For example, communications systems 718 can include one or more transceivers, one or more communication chips and/or chip sets, and so on. In a more particular example, communications systems 718 can include hardware, firmware, and/or software that can be used to establish a Wi-Fi connection, a Bluetooth connection, a cellular connection, an Ethernet connection, and so on.

In some embodiments, memory 720 can include any suitable storage device or devices that can be used to store instructions, values, data, or the like, that can be used, for example, by processor 712 to present content using display 714, to communicate with one or more computing devices 650, and so on. Memory 720 can include any suitable volatile memory, non-volatile memory, storage, or any suitable combination thereof. For example, memory 720 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 720 can have encoded thereon a server program for controlling operation of server 652. In such embodiments, processor 712 can execute at least a portion of the server program to transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone), and so on.

In some embodiments, the server 652 is configured to perform the methods described in the present disclosure. For example, the processor 712 and memory 720 can be configured to perform the methods described herein (e.g., the method of FIG. 2, the method of FIG. 5).

In some embodiments, data source 602 can include a processor 722, one or more data acquisition systems 724, one or more communications systems 726, and/or memory 728. In some embodiments, processor 722 can be any suitable hardware processor or combination of processors, such as a CPU, a GPU, and so on. In some embodiments, the one or more data acquisition systems 724 are generally configured to acquire data, images, or both, and can include a medical imaging system (e.g., an MRI system, a CT system). Additionally or alternatively, in some embodiments, the one or more data acquisition systems 724 can include any suitable hardware, firmware, and/or software for coupling to and/or controlling operations of a medical imaging system. In some embodiments, one or more portions of the data acquisition system(s) 724 can be removable and/or replaceable.

Note that, although not shown, data source 602 can include any suitable inputs and/or outputs. For example, data source 602 can include input devices and/or sensors that can be used to receive user input, such as a keyboard, a mouse, a touchscreen, a microphone, a trackpad, a trackball, and so on. As another example, data source 602 can include any suitable display devices, such as an LCD screen, an LED display, an OLED display, an electrophoretic display, a computer monitor, a touchscreen, a television, etc., one or more speakers, and so on.

In some embodiments, communications systems 726 can include any suitable hardware, firmware, and/or software for communicating information to computing device 650 (and, in some embodiments, over communication network 654 and/or any other suitable communication networks). For example, communications systems 726 can include one or more transceivers, one or more communication chips and/or chip sets, and so on. In a more particular example, communications systems 726 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 728 can include any suitable storage device or devices that can be used to store instructions, values, data, or the like, that can be used, for example, by processor 722 to control the one or more data acquisition systems 724, and/or receive data from the one or more data acquisition systems 724; to generate images from data; present content (e.g., data, images, a user interface) using a display; communicate with one or more computing devices 650; and so on. Memory 728 can include any suitable volatile memory, non-volatile memory, storage, or any suitable combination thereof. For example, memory 728 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 728 can have encoded thereon, or otherwise stored therein, a program for controlling operation of data source 602. In such embodiments, processor 722 can execute at least a portion of the program to generate images, transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the present disclosure.

1. A method for generating a radiation treatment plan for a spatially fractionated radiation treatment, comprising:
accessing medical imaging data of a subject with a computer system;
accessing dose vertex constraint data with the computer system, the dose vertex constraint data comprising spatial constraints on potential dose vertex locations;
accessing dose vertex objective data with the computer system, the dose vertex objective data comprising spatial preferences on potential dose vertex locations;
selecting a tumor region depicted in the medical imaging data using the computer system;
constructing a dose distribution with the computer system by generating and arranging dose vertices in the tumor region subject to the dose vertex constraint data and the dose vertex objective data; and
storing the dose distribution, by the computer system, in a radiation treatment plan for the subject.

2. The method of claim 1, comprising executing the radiation treatment plan with the computer system to control operation of a radiation treatment system to deliver therapeutic radiation to the subject.

3. The method of claim 1, wherein selecting the tumor region comprises generating a plurality of tumor regions and selecting one of the plurality of tumor regions capable of containing a planned number of dose vertices.

4. The method of claim 3, wherein the plurality of tumor regions is generated by the computer system using a clustering algorithm.

5. The method of claim 4, wherein the clustering algorithm is a hierarchical clustering algorithm.

6. The method of claim 3, wherein selecting the tumor region comprises fitting a geometric arrangement within each of the plurality of tumor regions and selecting the one of the plurality of tumor regions in which the geometric arrangement fits.

7. The method of claim 1, wherein the dose distribution is generated using a Monte Carlo simulation based on the dose vertex constraint data and dose vertex objective data.

8. The method of claim 7, wherein the Monte Carlo simulation comprises:
generating an initial dose distribution;
generating an intermediate dose distribution by randomly repositioning dose vertices in the initial dose distribution subject to the dose vertex constraint data and dose vertex objective data;
generating an updated dose distribution by adding an additional dose vertex to the intermediate dose distribution subject to the dose vertex constraint data and dose vertex objective data; and
storing the intermediate dose distribution as the dose distribution when the additional dose vertex is not capable of being added to the dose distribution subject to the dose vertex constraint data and dose vertex objective data and storing the updated dose distribution as the dose distribution when the additional dose vertex is capable of being added to the dose distribution subject to the dose vertex constraint data and dose vertex objective data.

9. The method of claim 1, wherein the dose vertex constraint data comprises spacing constraints of the dose vertices in the dose distribution.

10. The method of claim 9, wherein the spacing constraints comprise at least one of:
a minimum distance of the dose vertices from a surface of the tumor region;
a minimum distance of the dose vertices from an organ at risk (OAR);
a minimum center-to-center spacing between the dose vertices;
a minimum longitudinal separation between the dose vertices; or
a Euclidean distance between the dose vertices sharing a same z-coordinate.

11. The method of claim 10, wherein the minimum distance of the dose vertices from a surface of the tumor region is greater than or equal to 0.5 cm plus a dose vertex radius.

12. The method of claim 10, wherein the minimum distance of the dose vertices from an organ-at-risk is greater than or equal to 1 cm plus a dose vertex radius.

13. The method of claim 10, wherein the minimum center-to-center spacing between the dose vertices greater than or equal to 2 cm.

14. The method of claim 10, wherein the minimum longitudinal separation between the dose vertices is greater than or equal to 2 cm.

15. The method of claim 10, wherein the Euclidean distance between the dose vertices sharing a same z-coordinate is greater than or equal to 6 cm.

16. The method of claim 1, wherein the dose vertex objective data comprises a preference for centrally locating the dose vertices within the tumor region.

17. The method of claim 16, wherein the dose vertices are located subject to the dose vertex objective data based on a difference in a minimum distance to a center of mass between candidate dose vertex locations.

18. The method of claim 1, wherein at least one of the dose vertex constraint data or the dose vertex objective data comprise user preferences for at least one of dose vertex distances from organs-at-risk (OARs); minimizing dose received by OARs; or optimizing a relationship between dose to targets, tumor, and OARs.
